(19) United States
(12) Patent Application Publication
(10) Pub. No.: US 2021/0181212 A1
(43) Pub. Date: Jun. 17, 2021

(54) Early Stage Detection for Alzheimers and other Autoimmune Diseases

(71) Applicant: Richard Postrel, Miami Beach, FL (US)

(72) Inventor: Richard Postrel, Miami Beach, FL (US)

(21) Appl. No.: 17/187,880

(22) Filed: Mar. 1, 2021

Related U.S. Application Data
(63) Continuation-in-part of application No. 15/725,872, filed on Oct. 5, 2017.
(60) Provisional application No. 62/450,328, filed on Jan. 25, 2017.

(51) Int. Cl. G01N 33/84; G01N 33/497

(52) U.S. Cl. CPC G01N 33/84; G01N 2333/435; G01N 2800/00; G01N 33/497

(57) ABSTRACT

The present invention describes a non-invasive system and method for detecting early stage Alzheimer's and other autoimmune diseases associated with neurological deficits, including Multiple Sclerosis and Parkinson's Disease. By analyzing the volatile organic compounds (VOCs) found in the otic canal either in gaseous form or as what is commonly known as “earwax”, the current invention discloses how these disease signatures/profiles are illustrative of the presence or absence of a particular disease.

The present invention describes a non-invasive system and method for detecting early stage Alzheimer's and other autoimmune diseases associated with neurological deficits, including Multiple Sclerosis and Parkin's Disease. By analyzing the volatile organic compounds (VOCs) found in the otic canal either in gaseous form or as what is commonly known as “earwax”, the current invention discloses how these disease signatures/profiles are illustrative of the presence or absence of a particular disease.

VOCs are significant in the early detection and diagnosis of diseases of the nervous system, particularly those that originate in the brain, where the blood-brain barrier or the brain, itself can be an impediment to diagnosing a specific disease. VOC compounds signify metabolic responses that can serve as specific signatures to a particular neurodegenerative disease including, but not limited to: Multiple Sclerosis, Alzheimer's and Parkinson's Disease, etc.

VOC compounds which represent a particular signature or profile of a disease are comprised of small molecules that freely cross the blood brain barrier and thus are accessible and available for analysis. These compounds are products of the metabolic activities (healthy and diseased) within the brain for all individuals and their patterns of VOCs can be associated with a specific disease or a class of disease. The individual compounds each have an identifiable impact on one or more sensor surfaces. By analyzing across an array of sensors, the current invention teaches how to identify patterns of interactions that with high sensitivity and reliability indicate the presence of, absence of, or stage of, a particular disease with high sensitivity and specificity.

All diseases express characteristic symptoms that may change during disease inception and progression. But all disease symptoms have, as their basis, at least one factor that alters metabolism within cells leading to multiple compensating responses that eventually present as symptoms. These metabolic alterations involve modified or alternate biochemical reactions. The disease-associated patterns of altered or compensating metabolic reactions produce a signature pattern of biochemical reaction products and byproducts that include VOCs which may be analyzed to indicate disease and to differentiate between diseases.

The basic concept underlying looking at VOCs is supported in multiple peer reviewed journal articles. The world wide web also has similar disclosures. For example, in a 2019 article, Tridedi, et al. disclose a set of volatile biomarkers specific to Parkinson's Disease.¹ Skin swabs were collected from the upper back areas of Parkinson's Disease patients and a group of controls. The study highlighted detected levels of artemisinic acid, dodecane, eicosane, hexyl acetate, hippuric acid, octacosane, octadecanal, octanal, and perillic aldehyde using thermal desorption-gas chromatography-mass spectrometry (TD-GC-MS) analysis.

A 2017 paper reported results relating to diagnosis and classification of a plurality of diseases following analysis of exhaled breath.² The 17 diseases with reported results were: lung cancer, colorectal cancer, head and neck cancer, ovarian cancer, bladder cancer, prostate cancer, kidney cancer, gastric cancer, Crohn's disease, ulcerative colitis, irritable bowel syndrome, idiopathic Parkinson's, atypical Parkinsonism, multiple sclerosis, pulmonary arterial hypertension, pre-eclampsia, and chronic kidney disease. 2-ethylhexanol, 3-methylhexane, 5-ethyl-3-methyloctane, acetone, ethanol, ethyl acetate, ethylbenzene, isononane, isoprene, ¹ Discovery of Volatile Biornarkers of Parkinson's Disease from Sebum. Drupad K. Trivedi, Eleanor Sinclair, Yun Xu, Depanjan Sarkar, Caitlin Walton-Doyle, Camilla Liscio, Phine Banks, Joy Milne, Monty Silverdale, Tilo Kunath, Royston Goodacre, and Perdita Barran. ACS Cent. Sci. 2019, 5, 4, 599-606. Mar. 20, 2019. https:Thoi.org/10.1021/acscentsci.8b00879.² Diagnosis and Classification of 17 Diseases from 1404 Subjects via Pattern Analysis of Exhaled Molecules. Morad K. Nakhleh†, Haitham Amal, Raneen Jeries, Yoav Y. Broza, Manal Aboud, Alaa Gharra, Hodaya Ivgi, Salam Khatib, Shifaa Badarneh, Lior Har-Shai, Lea Glass-Marmor, Izabella Lejbkowicz, Ariel Miller, Samih Badarny, Raz Winer, John Finberg, Sylvia Cohen-Kaminsky, Frédéric Perros, David Montani, Barbara Girerd, Gilles Garcia, Gérald Simonneau, Farid Nakhoul, Shira Baram¶, Raed Salim¶, Marwan Hakim, Maayan Gruber, Ohad Ronen, Tal Marshak, Ilana Doweck, Ofer Nativ, Zaher Bahouth, Da-you Shi, Wei Zhang, Qing-ling Hua, Yue-yin Pan, Li Tao, Hu Liu, Amir Karban, Eduard Koifman, Tova Rainis, Roberts Skapars, Armands Sivins, Guntis Ancans, Inata Liepniece-Karele, Ilze Kikuste, Ieva Lasina, Ivars Tolmanis, Douglas JohnsonOrcid, Stuart Z. Millstone, Jennifer Fulton, John W. Wells, Larry H. Will, Marc Humbert, Martis Leja, Nir Peled, and Hassam Haick Orcid. ACS Nano 2017, 11, 1, 112-125. Dec. 21, 2016. https://doi.org/10.1021/acsnano.6b04930.

nonanal, styrene, toluene, and undecane were identified as being present in amounts that significantly differed in a disease from control groups and/or the other diseases. An “artificially intelligent nanoarray that is based on chemiresistive layers of molecularly modified gold nanoparticles and random network of single-wall carbon nanotubes” and GC-MS were used in analysis and characterization.

Another disease, Alzheimer's Disease (AD) tested the hypothesis “that dysregulation in energy use, mitochondrial abnormalities, oxidative stress, and neuroinflammation that occur with aging are contributing factors to the pathophysiology of AD.”³ In a rat model, an array including 3 VOC sensor elements butylated hydroxytoluene (BHT), pivalic acid and 2,3-dimethylheptane identified rats with an AD modeled mutation.

Thus, the concept that VOC analysis can be reliable in the diagnoses of diseases, generally, and specifically for neurological disease and autoimmune disease, is accepted in the art.

The present invention builds on these findings and features two improvements in sample analysis. The two means for reliable sample analyses and disease diagnoses disclosed as parts of the present invention feature a first means of analyzing a sample in the gas state and a second means of analyzing a sample in a solid or semi-solid state.

A primary site for sampling is the otic canal. The volume of gas within the canal turns over slowly. This slow turnover allows concentrations of volatile off gassing from the walls of the canal and the eardrum to achieve a semi-equilibrium state. That is a collection of off gasses emitted over time from the body and less contaminated that gasses that might be sampled off another body surface, such as forearm, armpit, torso, etc. The otic canal is also a source or earwax, a protective secretion lining the canal. Earwax is a source of multiple volatile organic compounds (VOCs) that may be assessed to evaluate metabolism within the body and especially in the head and brain area. Samples may be obtained bilaterally using an earwax ³ Detection of presymptomatic Alzheimer's disease through breath biomarkers. Shadi Emam, Mehdi Nasrollahpour, Bradley Colarusso, Xuezhu Cai, Simone Grant, Praveen Kulkarni, Adam Ekenseair, Codi4 Gharagouzloo, Craig F. Ferris, Nian-Xiang Sun. Alzheimer's & Dementia: Diagnosis, Assessment & Disease Monitoring. 14 Oct. 2020. https://doi.org/10.1002/dad2.12088

removal tool or swab when desired to potentially differentiate severity of disease relating to the left and right sides or brain hemispheres.

VOC detection devices have been described in detail, for example, in U.S. patent application 63/017,693 filed Apr. 30, 2020; the disclosures of which are hereby included in their entireties by reference. Developers are continuously improving the capabilities of electronic noses using tried and true sensors such as metal oxides. See, for example, “Robust and Rapid Detection of Mixed Volatile Organic Compounds in Flow Through Air by a Low Cost Electronic Nose”, by Huang and Wu, published Aug. 21, 2020 wherein acetone, ethanol and isopropyl alcohol were detection targets, indicating that cross referencing a plurality of sensors within an analytical algorithm appears to offer detection advantages.

A preferred sensing device is a high sensitivity device that features single walled carbon nanotubules (SWNTs) as a surface to interact with the VOC compounds being evaluated. Other embodiments may feature graphene or synthetic polymers to similar effect. SWNTs and other carbon substrates such as thin or single layer graphene provide both a large surface to volume ratio to facilitate sensor-molecule interaction, and electrical conductivity that facilitate signal transduction. In the April 30 patent referenced above, nano-sensor elements (NSEs), each including at least one sensing surface, are capable of, for example, of field-effect transistor (FET) or other physico-electrical property/activity. Such structures include, but are not limited to: semi-conducting nano-wires, carbon nano-tubes—including single-wall carbon nano-tubes, chitosan-cantilever based, synthetic polymers—including dendrimers, plasmon resonance nano-sensors, Förster resonance energy transfer nano-sensors, paramagnetic compounds, surface active crystals, vibrational phonon nano-sensors, magnetically resonant compositions, optical emitting or transforming compositions, optical frequency (or wavelength) based nano-sensors (sensitive to photon transmittance, absorption, reflection, energy modulation, etc.).

One preferred format of the present invention may feature “chips” with modular nano-sensing elements (or nano-sensor element (NSE) that are independently maintained at a fixed, fluctuating, stochastic, alternating, discontinuous or flashing feeder power supply. The outputs of each NSF may be individually wired to a dedicated data transducer or a selection of sensor outputs may use a common carrier circuit and thus be “averaged”. In some embodiments, a simpler circuitry may involve multiple elements feeding a single output that may sum the outputs to deliver an average reading. When one or more of the “averaged” sensors is turned off or powered down, the average will not include output from these one or more powered down sensors. When input sensors are powered individually, for example, in a cycling pattern when only one (or a selected portion) of the input electrodes being charged, averaged outputs synchronized with the timing of input charging can thus provide data from individual channels.

The single output may connect and thereby collect data signal from any desired fraction of elements. For example, a single output may receive signal from all elements on a chip, half the elements on a chip, one-third the elements on a chip, a quarter the elements on a chip, a fifth the elements on a chip, and so on, for example, ⅙, 1/7, ⅛, 1/9, 1/10, 1/12, 1/20, 1/25, 1/33, 1/50, 1/100, etc. Any output may be associated with 1, 2, 3, 4, 5, 6, 7, 8, 9, 10, 11, 12, 13, 14, 15, 16, 17, 18, 19, 20, 21, 22, . . . , 24, . . . , 32, . . . , 48, . . . , 50, . . . , 64, . . . , 96, 100, . . . , 128, . . . , 200, . . . , 250, . . . , 256, . . . , 500, . . . , 512, . . . , 1000, . . . , 1024, . . . , 2048, . . . , 4096, . . . , 5000, . . . , 8192, . . . , 10,000 (10⁴), . . . , 16,384, . . . , 2¹⁵, . . . , 2¹⁶, . . . , 10⁵, . . . , 2¹⁷, . . . 2¹⁸, . . . 2¹⁹, 10⁶, . . . , 2²⁰°, . . . total number of sensors on a chip which may vary with time or programmed instructions. The precise count of sensor elements associated with any output in general is a design feature and does not define operative functions of the invention. The counts specifically exemplified above are exemplary low numbers of sensors that may feed an output and higher numbers common in conventional plate assays and powers of 2 and 10 frequently used or approximated in biological or chemical science or physics or electronics.

When connected to multiple elements, the output may average output signals from each, and modulate weightings of elements in an average or in contribution to signature formation. With fluctuating or non-constant inputs, weighting is also controllable. For example, in an extreme sense a stochastic or alternating input, when alternated to off that element's output will report a zero weighting, or a fluctuating or stochastic feed can serve to physically, rather than mathematically, control the weighting output. The designer and/or operator will have options for mathematical/algorithmic or physical/electrical weighting of each NSE input to the data analysis. A group of elements may therefore receive the same feeder voltage, or the feeders may be independently controlled.

Instruction to or control of the system may be through information encoded on a sample package, information encoded on a sensor chip, from a user interface, information provided remotely by machine or active user, or information encoded within the device. For example, samples may be encoded with a shape or mass signal. A sample having a given shape would instruct the device to proceed with the assay that the software associates with that shape. In addition to shape, sample cartridge mass may be instructive as to the sample mass itself or may, perhaps distinguishing a smaller or a larger sample, instruct processing of the sample to allow access at controlled volume or feed rate of the VOCs into analysis. An optically readable signal, (color, transparency, bar code, text, etc.) an electronically accessible signal (RFID, memory chip or drive, etc.), a magnetic signal, etc., are also useable in controlling the device. Specific control can be through a large variety of means and is not generally to be considered as limiting the invention. The signal embedded itself may be adequate to program the relevant machine cycles or may instruct the machine to access further instructions for example, in machine archives or at a remote location. A device may cycle through one or a plurality of signals as directed or required. Chips may be interchangeable and be encoded using signals analogous to those discussed above relating to sample cartridges.

Such device is preferably extremely compact. In a preferred embodiment, an otic gas sample is analyzed in a probe fixture, e.g., a device similar in shape to an otoscope, that collects and analyzes an otic gas sample. Data may he stored and analyzed within the device and/or transmitted to an accessory device for data retention and analysis. In other embodiments, a gas sample or a plurality of samples may be collected for delivery into a remote device by direct streaming from the canal probe to an assay analysis component or may be captured in a pod with physical delivery of the pod to an analytical device.

When earwax is used, a remote device may be preferred. A remote device into which gas samples are delivered and a remote device that analyzes earwax may be identical, with only a difference in how sample gas is delivered to the sensing block, e.g., array of decorated SWNTs. One form of sensor selected by the practitioner may be a nano FET. A gas may be directly injected using, e.g., pressure difference, to convectively deliver the sample. An earwax removal tool or a swab may be delivered to a location accessible to the sensing block for off gassing to the block. A solvent or a sequence of solvents may be used to extract substances from the wax for delivery through the sensing chamber apparatus. Alternatively, a flushing with a solvent, e.g., water, alcohol, peroxide, mild acid, etc., may extract coating from the canal surface without use of solid earwax collection device. The solvent in the extraction active at the time of reading may be taken as a significant factor in crafting a standardized signature. For example, a polar and non-polar solvent may be used in sequence to assist the device in characterizing compounds that interact with any individual element or group of sensing elements.

Off gassing may be accomplished by passage of time, but more preferably by thermally exciting the VOCs in the wax to promote delivery to and contact with one or more sensing elements. Any manner of controlled heating is acceptable. Thermal excitation may similarly be applied to promote deliver of VOCs to and interaction with a sensing element, block, chip, etc.

The sensing component or block, which may comprise a chip or a plurality of chips, monitors electrical changes in response to proximity of a VOC to a sensor element of the component. Numerous sensor elements are used to differentiate between VOC molecules interacting with the plurality of elements. A pattern of element-VOC interactions is collected and analyzed to form a signature. Sensing elements may be maintained at a static temperature or may be heated or cooled during an analysis session. Instantaneous temperature of the sensing component and/or ambient vapor may be a factor in or a part of the formed signature. Individual elements may be heated individually. Zones or volumes within the sensing volume may be excited, e.g., by light, ion beam, etc. to enable additional factors to be included in a signature.

The signatures associated with different diseases may include common elements, including one or a group of elements maintaining similar ratios. For example, an autoimmune disease may provoke development of one or more VOCs rooted in the immune process. A signature may be identified as associated with autoimmunity even if the auto-target is not identified. Immune suppression may relieve symptoms by reduce immune attack even in the absence of specific identification or characterization of the disease. A signature relevant to a disease may include extractable features associated with a disease class. The disease signature may share components with several disease or classes of diseases. Disease distinguishing features may be a single or plurality of characterizing VOCs. A ratio between two or more VOCs, rather than simple presence or absence detected in the sample, may serve to indicate presence of disease or to differentiate between diseases.

In autoimmune disease, a receptor targeted by the disease may increase and/or decrease certain activities within a cell under control of the receptor. In the nervous system, there may be a cascading effect wherein a decrease or increase of a neurotransmitter may affect activities of downstream cells or upstream cells involved in a feedback loop, Metabolites of these cells may be a part of a signature associated with the autoimmune disease targeting that receptor or others in the neuropathway(s).

When a cell protein, not necessarily an active receptor, comes under attack, mechanics of the cell plasma membrane may be affected. For example, if a white cell membrane were to be made less deformable, flow of that cell through arterioles or capillaries may aggravate endothelial cells whose VOCs may become part of that disease signature.

The outcome of early methodology of the present invention is a library of signatures. Signatures may be associated with a class (e.g., autoimmune) of diseases, a cell type associated with the disease, a tissue or organ associated with a disease, a recognized disease(e.g., Lupus, Alzheimer's, Parkinson's, Multiple Sclerosis, H₁N₁ flu, gall bladder, etc.), a stage of disease, e.g., pre-symptomatic, mufti-location, etc.). A patient's signature is compared to library signatures to suggest diagnosis and/or treatment specific for that patient.

### EXAMPLES

A panel of patients is chosen. Informed consent is obtained. Patients are associated with one or more disease(s). When patients have agreed, both tool or swab derived samples (earwax) and otic canal gasses are collected. Samples are analyzed and a signature pattern output is obtained. It is not essential to identify any specific VOC, the sensing pattern obtained by passing the sample over the block provides sufficient distinguishing data even when the chemical structure of the VOC is unknown. Patients with a particular diagnosis are associated with that disease. Patients without that diagnosis (but possibly, and most likely, with a diagnosis for one or more other disease(s) can serve as control for each disease in the panel of patients.

When gas is targeted for analysis, a gas sample outside the otic canal may be analyzed as a control factor. The sensor device used for obtaining and analyzing otic gas may be activated outside the canal to collect control samples. For example, a sample of gas from the auricular area, the external meatus, etc., may serve to control for ambient gases the subject may be immersed in.

The data are fed into a processor either in the collection device itself or an associated component which applies artificial intelligence or machine learning to identify portions of each patient's signature may be associated with a particular diagnosis. In some circumstances a part of the signature, e.g., a ratio of VOC A to VOC B may be similar between a plurality of diseases. But other parts of the signature may differentiate between the diseases. A library of signature patterns is thus collected with characteristic signature elements being associated with a disease as signature for that disease. Earwax and otic canal gasses are separately analyzed but may be correlated or cross-referenced. Left- and right-side readings are cross-referenced and correlated when possible. Differences may be indicative of disease differences between hemispheres, circulatory aberrations, previous injury, sleep positions, headset wearing, etc. Data may show that left-right differences can provide information suggesting lesion location, or may suggest preferences for using the right or left ear for testing depending on the subject's behaviors, habits, and activities.

A second group of patients is similarly evaluated to confirm or adjust disease signatures. The system is then used for diagnosing patients. In preferred practice, over a period of years, the data are periodically reevaluated and refined. For example, patients who are diagnosed with a disease months or years after the initial signature development may have their data reevaluated for potential indication of a pre-disease state or early disease detection. Having a signature library available, a patient comes to clinic and as a part of screening has an otoscope like device inserted in the ear canal. This exemplary device actually includes an otoscope function incorporating a light and a view-port. The medical provider uses the optics of the otoscope to center the device within the canal. A gas sample is drawn into and analyzed in the otoscope device. The device communicates the patient's otic gas signature electronically to a home device which displays and/or prints out a report. A clinician then counsels the patient with emphases on current and developing disease(s) that are indicated or suggested through the device's comparison of the patient's VOC signature to the signature library for diseases.

An alternative embodiment format draws otic canal gases through the canal inserted portion of the device into an accessory device containing one or more sensing blocks. The gas replacing the gas removed by the device may be ambient air or a selected gas or mixture of gases. For example, an inert gas (which may or may not be a noble gas) in provided at a temperature or range of temperatures to optimize testing protocols, e.g., for speed, patient comfort, quality of results.

As the gases are drawn through the accessory device the depth of the otic probe may be adjusted. A low-volume transit tube, e.g., short with small inner diameter, allows obtention of results at a quicker pace and decreases temperature effects from gases flowing into the ear canal when these flows are not otherwise controlled. To the extent that colder air may be annoying to the subject, a heating coil or feeding line may reduce the irritative cold sensation. Slightly warming the air may also be advantageous for subliming or evaporating additional VOCs. Humidity may also be a controlled testing parameter. Polar vapors, water or otherwise, may encourage release or VOCs from the otic coating. Volunteer subjects or patients of different sizes, genders, races, and cultures are tested using probes with flowing gas.

The art mentioned above employed different means for assaying volatile compounds. The different means would be expected to have different sensitivities to different VOCs. In this current example, temperature is a variable that can change the signature profile. Accordingly, a VOC pattern associated with a disease, e.g., Alzheimer's Disease obtained, for example through GC-MS, cannot be assumed to be the same pattern when assayed with another sensor format. Thus, signatures should be clearly identified with the process under which they were obtained.

Nano FETs and other nano-sensor formats generally operate by changing electrical properties as a substance comes in close proximity to the sensor. The interaction between electrons of the sensed molecule and the sensor surface perturbs the steady state of that surface to elicit its signal. The altered distribution of electrons induced by a proximal molecule, (depending on the design of the nano-sensor) changes one or more electrical properties, e.g., impedance, resistance-conductivity, capacitance, inductance, etc. and thus the physical movement of a detectable particle, e.g., an electron, a photon, etc.

Specificity of coordination (interaction) between sensor surface and VOC molecule may be provided by functionalizing or decorating the carbon gate electrode. For example, many sequences of nucleic acid such as DNA or RNA will stringently coordinate or bind with the SWNT structure. These nucleic acids may be naturally occurring or synthetic. The ringed structures of the nucleic acids or other molecules such as peptides containing a large fraction of ringed structures associate strongly with the nanotubular structures. These functionalizing, or decorating, additions to the SWNTs serve to selectively capture proximal molecules. When the chemical geometry is changed, the gating characteristic of the associated carbon bridging the input and output electrodes is modulated. Differently decorated or heated elements respond differently different proximal VOC. A single element may be associated with a single sequence or a plurality of functionalizing sequences. Output characteristics of gating in response to one or more gaseous compounds, e.g., VOCs are then collated into a data library. When that NSE responds in the same manner, presence of the VOC is confirmed. Stringent selection of element functionalizations, and subsequent application of the controllable assay variables can optimize certainty of VOC identification at a desired level, for example, increasing manipulation of the variable parameters can achieve certainty of 99+%. In special circumstances, for example to develop rapid profiling of a new VOC signature (i.e., pathogen), a simplified screening protocol or developmental process may begin with a lower level of certainty, e.g., 85%, 95%, etc. Subsequent refinements then could be applied to raise the level of certainty until reaching a mathematical and chemical sensitivity to an acceptable level, e.g., a 99+% certainty while also minimizing false positives.

A single element may be capable of indicating the presence of more than one compound. For example, similar compounds may not be distinguished in their association/coordination with the element surface and therefore may in certain circumstances produce indistinguishable signals on their own. But the single element may, for example, in conjunction with one or more other elements provide definitive results with respect to the VOCs that may interact with any one element. Alternatively, the single element when operated at a different temperature, voltage or other variable may distinguish between the different compounds binding the element under static conditions. The discussion above describing the variable inputs and input patterns and different resulting outputs relates to such differentiation capabilities.

Since the manner through which a signature is obtained is determinative of the signature outcome a different assay technique thus requires validation processes to form and confirm VOC signatures for each disease of interest. While animal models may be illustrative, human data are preferred for better relevance to human diseases.

1. A method for assessing for a disease or condition, said method comprising:
i) collecting a sample from the otic canal;
ii) analyzing content of at least one VOC in said sample;
iii) forming a signature indicative of said analyzed content;
iv) comparing said signature to a library of signatures associated with a disease or condition; and
v) acknowledging similarities between said signature formed in iii) and one or a plurality of library signatures.

2. The method of claim 1 wherein said sample comprises a gaseous sample.

3. The method of claim 1 wherein said sample comprises a coating on the wall of the otic canal.

4. The method of claim 1 wherein said sample comprises earwax.

5. The method of claim 2 wherein analyzing said gaseous sample comprises:
inserting a probe into said otic canal;
contacting a sensing surface of said probe with otic canal gas;
collecting data resulting from said contacting;
forming a signature by analyzing said data resulting from said contacting;
comparing said signature to a library of signatures associated with a disease or condition; and
acknowledging similarities between said signature formed by analyzing said data resulting from said contacting and one or a plurality of library signatures.

6. The method of claim 2 wherein collecting a sample from the otic canal comprises:
inserting a probe into said otic canal; and
drawing gas from said otic canal into said probe.

7. The method of claim 6 wherein said collecting a sample from the otic canal comprises:
causing said gas to flow from said probe into a device that analyzes VOCS.

8. The method of claim 7 wherein said device that analyzes VOCs comprises a nanosensing element.

9. The method of claim 6, wherein said collecting a sample from the otic canal comprises:
causing said gas to flow into a cartridge; sealing said cartridge; and
delivering said sealed cartridge for said analyzing.

10. The method of claim 3 wherein said collecting a sample from the otic canal comprises physically removing said coating using a solid support.

11. The method of claim 10 wherein said solid support is selected from the group consisting of: an earwax removal tool and a swab.

12. The method of claim 10 further comprising inserting said solid support into a chamber configured to deliver said sample for said analyzing.

13. The method of claim 12 further comprising thermally exciting said sample on said solid support to promote movement of VOCs and subsequent contact of at least one VOC with at least one sensing element.

14. The method of claim 10 further comprising solvent extracting said coating from said solid support.

15. The method of claim 14 further comprising thermally exciting said extracted sample to promote movement of VOCs and subsequent contact of at least one voe with at least one sensing element.

16. The method of claim 3 wherein said collecting a sample from the otic canal comprises extracting with a liquid solvent.

17. The method of claim 16 further comprising thermally exciting said extracted sample to promote movement of VOCs and subsequent contact of at least one VOC with at least one sensing element.

18. The method of claim 2 wherein analyzing said gaseous sample is performed by contacting at least one nanosensing element with gas in the otic canal.

19. A method for forming a library of signatures associated with a first disease or condition, said method comprising:
i) assembling a cohort of informed subjects;
ii) associating each member of said cohort with any or all diseases said each member has been diagnosed to have;
iii) collecting a sample from the otic canal of each member;
iv) analyzing content of at least one VOC in said sample;
v) forming a signature indicative of said analyzed content;
vi) identifying as a control member for said first disease or condition, a subject not associated with said first disease or condition;
vii) comparing a plurality of control members' VOC content for said first disease or condition with VOC content of each member diagnosed to have said first disease or condition;
viii) documenting differences in analyzed VOC content between control members and diagnosed members to form a signature associated with said first disease or condition.

20. The method according to claim 19 further comprising:
forming a library of signatures associated with a second disease or condition by applying vi), vii), and viii) to said second disease or condition.

21. The method according to claim 20 further comprising:
repeating the method of claim 20 to a plurality of second diseases or conditions;
compiling said libraries of signatures in a collection comprising a plurality of signatures associated with a plurality of diseases or conditions.
